(19) United States
(12) Patent Application Publication
(10) Pub. No.: US 2019/0110741 A1
(43) Pub. Date: Apr. 18, 2019

(54) LIFE RHYTHM MEASUREMENT SYSTEM AND LIFE RHYTHM MEASUREMENT METHOD

(71) Applicant: Hitachi-LG Data Storage, Inc., Tokyo (JP)

(72) Inventors: Yasuhiro ISHII, Tokyo (JP); Takahiro KOMAKI, Hitachi (JP)

(21) Appl. No.: 16/137,566

(22) Filed: Sep. 21, 2018

(30) Foreign Application Priority Data
Oct. 12, 2017 (JP) 2017-198673

(51) Int. Cl. A61B 5/00; A61B 5/11; A61B 5/107

(52) U.S. Cl. CPC A61B 5/4857; A61B 5/1115; A61B 5/1116; A61B 5/1077; A61B 5/746; G01S 17/42; A61B 5/4809; A61B 5/1128; A61B 5/0077; A61B 2505/07; A61B 2503/08; A61B 5/1118

(57) ABSTRACT

In the life rhythm measurement system, a distance measuring device measures 3D information (whether a posture corresponds to a standing posture, a sitting posture, or a lying posture, a movement amount, and absence information) of a measurement target person using a TOF sensor. A management device aggregates the 3D information measured by the distance measuring device for each unit time, estimates a life rhythm (a ratio of each posture per unit period, a movement amount, an absence time, a bedtime, a wake time, and a home return time) of the measurement target person from a 3D data for each time, and accumulates the estimated life rhythm in a storage device. The management device provides information on the life rhythm of the measurement target person accumulated in the storage device to a measurement requester.

### CLAIM OF PRIORITY

The present application claims priority from Japanese patent application serial No. JP 2017-198673, filed on Oct. 12, 2017, the content of which is hereby incorporated by reference into this application.

### BACKGROUND OF THE INVENTION

### (1) Field of the Invention

The present invention relates to a life rhythm measurement system and a life rhythm measurement method for measuring a life rhythm for a person.

### (2) Description of the Related Art

In recent years, for example, a demand for monitoring a living condition of an elderly person living alone, etc. from a remote place has been increasing. As a method of monitoring the elderly person, there is a method of installing a camera in a house of a target person to be watched and transmitting a photographed image to a remote place via the Internet to directly monitor a state of the target person (for example, see JP 2002-291057 A). In addition, there is a method of installing a motion sensor or a door opening/closing sensor in a house of a target person to be watched and detecting an in-house position of the target person to indirectly monitor a state of the target person from position information (for example, see JP 2005-115412 A). Further, a management device installed in the remote place corresponds to a system that issues an alarm when there is a change in the state of the target person.

### SUMMARY OF THE INVENTION

In the direct monitoring method using the camera as in JP 2002-291057 A, a camera image is transmitted without change, every movement of the target person is exposed, and the method is likely to be rejected by the target person to be watched in terms of privacy.

Meanwhile, in the indirect monitoring method using the motion sensor as in JP 2005-115412 A, a small animal such as a pet other than the target person is erroneously detected, and monitoring accuracy for a specific person decreases. In addition, even in the case of determining a behavior of a person based on opening/closing of a door, a usage situation of an electric kettle, etc., information about presence of someone at a point in time is merely obtained, and it is impossible to sufficiently meet a demand for knowing a life rhythm of the target person.

An object of the invention is to provide a life rhythm measurement system and a life rhythm measurement method for measuring a life rhythm of a measurement target person while protecting privacy of the measurement target person.

The invention is a life rhythm measurement system for measuring a life rhythm of a measurement target person, including a distance measuring device that measures 3D information of the measurement target person present in a specific living space, and a management device that aggregates the 3D information measured by the distance measuring device for each unit time, estimates the life rhythm of the measurement target person from a 3D data for each time, and accumulates the estimated life rhythm in a storage device, in which the management device provides information on the life rhythm of the measurement target person accumulated in the storage device to a measurement requester.

In addition, the invention is a life rhythm measurement method of measuring a life rhythm of a measurement target person, including a step of measuring 3D information of the measurement target person present in a specific living space, a step of aggregating the measured 3D information for each unit time, estimating the life rhythm of the measurement target person from a 3D data for each time, and accumulating the estimated life rhythm in chronological order, and a step of providing information on the accumulated life rhythm of the measurement target person to a measurement requester.

According to the invention, it is possible to provide a life rhythm measurement system and a life rhythm measurement method for measuring a life rhythm of a target person while protecting privacy of the target person.

### BRIEF DESCRIPTION OF THE DRAWINGS

These and other features, objects and advantages of the present invention will become more apparent from the following description when taken in conjunction with the accompanying drawings wherein:

FIG. 1 is a block diagram illustrating an overall configuration of a life rhythm measurement system;

FIG. 2 is a diagram illustrating a configuration of a time of flight (TOF) sensor;

FIG. 3A is a flowchart illustrating preprocessing in a TOF device;

FIG. 3B is a flowchart illustrating a state determination process;

FIG. 3C is a flowchart illustrating a person recognition process;

FIG. 3D is a flowchart illustrating a life rhythm measurement process;

FIG. 4A is a flowchart illustrating an hour-based data editing process;

FIG. 4B is a flowchart illustrating a day-based data editing process;

FIG. 4C is a flowchart illustrating a bedtime/wake time calculation process;

FIG. 4D is a flowchart illustrating a home return time calculation process;

FIG. 5A is a diagram illustrating an example of a screen showing a current state of a measurement target person in a room;

FIG. 5B is a diagram illustrating an example of a screen showing an hourly life rhythm of the measurement target person;

FIG. 5C is an example of diagram showing a daily life rhythm;

FIG. 5D is an example of diagram showing a weekly life rhythm;

FIG. 5E is an example of diagram showing a monthly life rhythm;

FIG. 6A is a diagram illustrating a content example of a non-waking-up mail; and

FIG. 6B is a diagram illustrating a content example of a non-home return mail.

### DETAILED DESCRIPTION OF THE EMBODIMENT

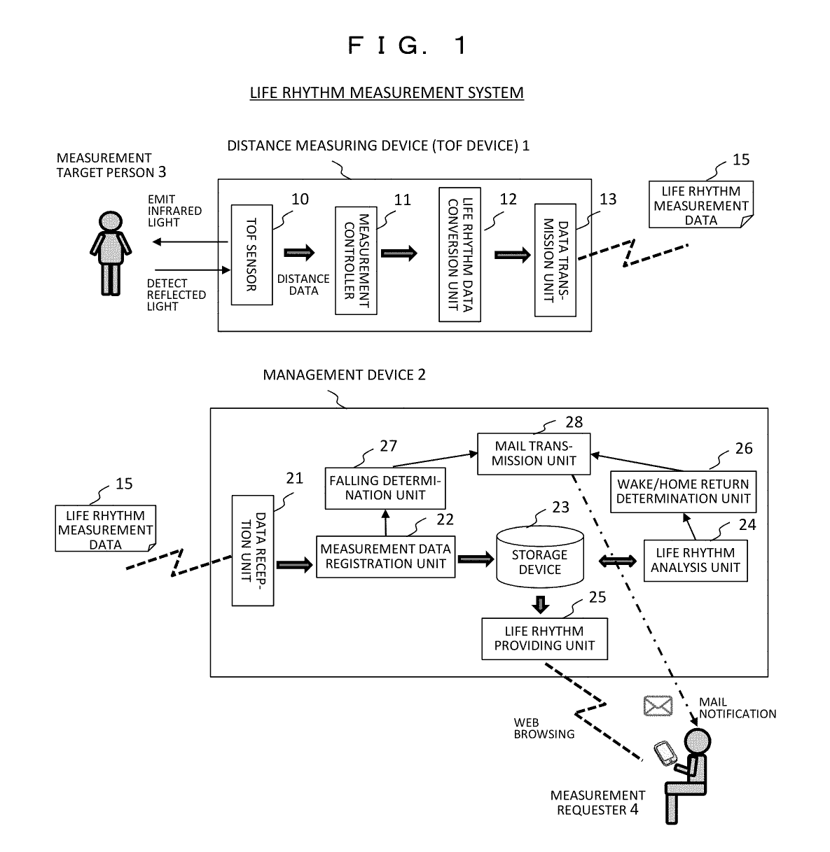

Hereinafter, an embodiment of the invention will be described with reference to drawings. FIG. 1 is a block diagram illustrating an overall configuration of a life rhythm measurement system. For example, the life rhythm measurement system includes a distance measuring device 1 that measures a living condition of a measurement target person 3 (target person to be watched) such as an elderly person living alone, and a management device 2 that estimates a life rhythm of the measurement target person 3 by analyzing measurement data. The management device 2 provides information on the life rhythm of the measurement target person 3 to a measurement requester 4 (monitoring requester), and issues a notification of an alarm when an abnormality is detected in the living condition of the measurement target person 3. In this way, the measurement requester 4 may check the life rhythm of the measurement target person 3 and take appropriate support measures as necessary. Incidentally, the measurement requester 4 is not limited to a person requesting monitoring and may correspond to a device (system). Hereinafter, an object referred to by “measurement requester” includes the device.

The distance measuring device 1 (hereinafter also referred to as a TOF device) includes a TOF sensor 10 that measures a distance to a person to be measured by a flight time of light. The TOF sensor 10 emits a laser beam and calculates an arrival time of light reflected by hitting an object, thereby obtaining a distance to the object. When a measurement region is divided in a lattice shape, and a distance to each lattice point is obtained, it is possible to obtain 3D distance data (3D point group image) such as a position, a height, a width, and a depth of an object in the region. Position information of a person in a living room is obtained by performing this operation with respect to the person. A measurement controller 11 controls measurement timing or a measurement range by the TOF sensor 10. A life rhythm data conversion unit 12 analyzes the 3D distance data (3D point group image), identifies that the person corresponds to the measurement target person 3 (hereinafter target person), and obtains information on a position, a posture (standing posture, sitting posture, lying posture), a movement amount, presence/absence of the target person in the living room. These pieces of information correspond to basic data for estimating the life rhythm of the target person, and thus are referred to as “life rhythm measurement data”. A data transmission unit 13 periodically transmits (for example, every minute) life rhythm measurement data 15 to the management device 2.

The management device 2 receives the life rhythm measurement data 15 transmitted from the distance measuring device 1 using a data reception unit 21, a measurement data registration unit 22 registers this data in a storage device 23, and the life rhythm measurement data 15 is accumulated in chronological order. A life rhythm analysis unit 24 aggregates the life rhythm measurement data 15 for each unit time (minute, hour, day, week, month, year, etc.), estimates bedtime, leave-time, etc. of the target person, and registers the estimated bedtime, leave-time, etc. in the storage device 23. In response to a request from the measurement requester 4, a life rhythm providing unit 25 provides information on the life rhythm of the target person by Web browsing, etc. Upon detecting an abnormality in a wake time or a home return time of the target person, a wake/home return determination unit 26 sends a mail to the measurement requester 4 via a mail transmission unit 28 to report the abnormality. Upon determining that the target person has fallen, a falling determination unit 27 sends a mail to the measurement requester 4 via the mail transmission unit 28 to report the abnormality.

Incidentally, in the life rhythm measurement system, the management device 2 has a mode (receiving hub scheme) in which the life rhythm measurement data 15 is received from each of a plurality of distance measuring devices 1 and collectively managed and a mode of exclusively managing the data by being connected to one distance measuring device 1. In the latter case, it is possible to adopt a configuration of installing in the vicinity of the distance measuring device 1 or a configuration as an integrated device. In addition, the storage device 23 in the management device 2 may be configured as an external device of the management device 2.

The above-described various processing operations in the distance measuring device 1 and the management device 2 are implemented by loading each of execution programs in a memory (not illustrated) and executing the program by a CPU. First, an operation of the distance measuring device (TOF device) 1 will be described in detail.

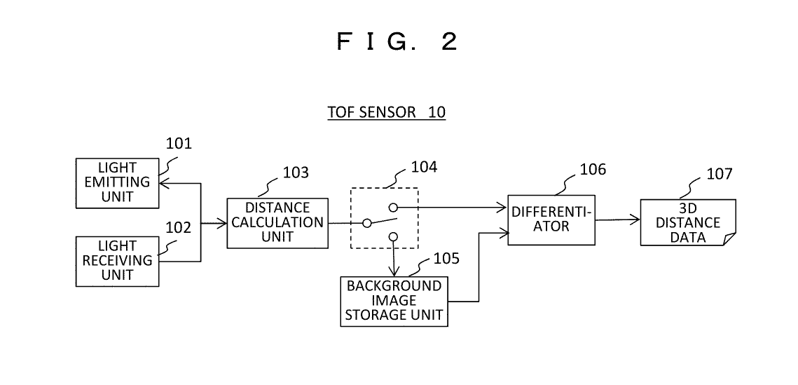

FIG. 2 is a diagram illustrating a configuration of the TOF sensor 10 in the TOF device 1. To measure a distance, the TOF sensor 10 includes a light emitting unit 101 such as a laser diode (LD) or a light emitting diode (LED) that irradiates infrared pulsed light and a light receiving unit 102 such as a CCD sensor or a CMOS sensor that receives pulsed light reflected from a subject. A distance calculation unit 103 drives the light emitting unit 101 and calculates a distance from a detection signal of the light receiving unit 102 to the subject. When a two-dimensional (2D) image of the subject is captured in the light receiving unit 102, the distance calculation unit 103 outputs distance data of the 2D image of the subject, that is, 3D distance data.

At the time of measuring a distance of a person, a background image in which the measurement target person 3 in the subject is not present is captured by a background image capturing unit 104 and stored in a background image storage unit 105. A differentiator 106 removes the background image from a subject image including the person to generate 3D distance data (3D point group image) 107 extracting the person.

For example, the measurement controller 11 controls the TOF sensor 10 to obtain 3D distance data of the measurement target person 3 in the living room every one second. For example, the life rhythm data conversion unit 12 converts the data into a standing posture time, a sitting posture time, a lying posture time, an absence time, and a moving distance per minute (a sum of moving distances in a horizontal direction for one second), and generates life rhythm measurement data together with latest information on a position, a height, and a posture of the target person. In addition, when a person other than the target person (for example, a visitor) is detected, the number and positions of visitors staying within one minute may be included in the life rhythm measurement data.

In this example, a measurement space is set to a living room in which the measurement target person 3 is mainly located. Therefore, when the person is in a bedroom or a toilet, it is determined that the person is absent. Incidentally, when the TOF device 1 (or the TOF sensor 10) is installed in the bedroom, a kitchen, a bathroom, the toilet, etc. other than the living room, and measurement data thereof is combined, it is possible to more specifically and accurately estimate the life rhythm of the target person.

The life rhythm measurement data 15 for each minute measured by the TOF device 1 is transmitted from the data transmission unit 13 to the management device 2. Thereafter, the TOF device 1 resets the measurement data and performs measurement for a subsequent minute.

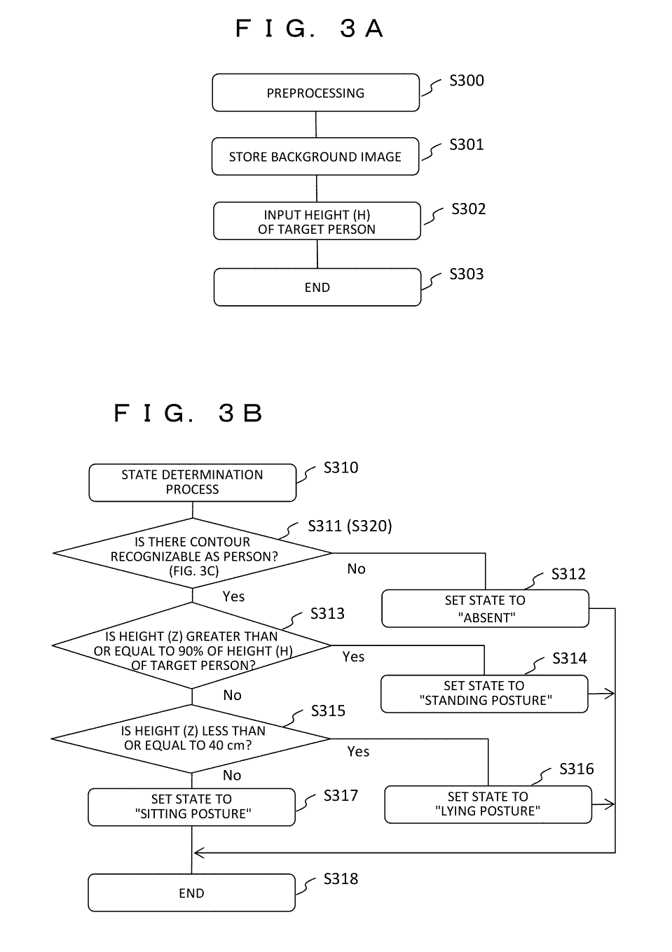

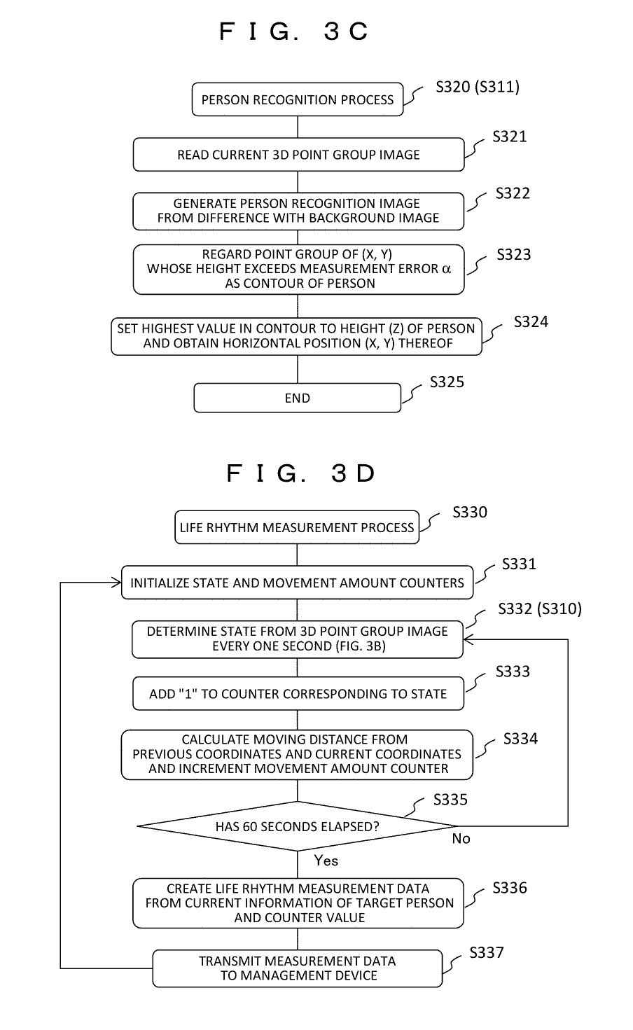

FIG. 3A to FIG. 3D illustrate various operation flows in the TOF device 1. FIG. 3A is a flowchart illustrating preprocessing (S300) in the TOF device 1. This process is executed by the measurement controller 11. First, 3D distance data (3D point group image) of a room without a person is obtained by the TOF sensor 10, and this data is stored in the background image storage unit 105 as a background image (S301). The obtained data corresponds to a 2D array indexed by a width (X axis) and a depth (Y axis) of the room, and each element of the array corresponds to a height (Z axis) of an object at a position (X, Y) thereof. Subsequently, a height (H) of the target person 3 is input (S302). For example, a highest point of an object entering a measurement range of the TOF sensor 10 in a first period (10 frames) is set to a value of a height (H).

FIG. 3B is a flowchart illustrating a state determination process (S310) in the TOF device 1. This process is to determine a current state (standing posture, sitting posture, lying posture, and presence/absence) of the target person 3 from the 3D distance data (3D point group image), and is executed by the life rhythm data conversion unit 12. First, it is determined whether there is a contour recognizable as a person in an image (S311), and a state is set to “absent” when the contour is not present (S312). Incidentally, a person recognition process will be described below (FIG. 3C). When the person is present, and a height (Z) thereof is greater than or equal to 90% (example) of the height (H) of the target person, the state is set to “standing posture” (standing state) (S314). When the height (Z) of the person is less than or equal to 40 cm (example), the state is set to “lying posture” (lying state) (S316). When the height (Z) of the person is in the middle, the state is set to “sitting posture” (sitting state) (S317). In this way, it is possible to determine the current state (posture, presence/absence) of the target person.

FIG. 3C is a flowchart illustrating a person recognition process (S320) and specifically illustrates the person recognition process (S311) of FIG. 3B. A current 3D point group image is read by the TOF sensor 10 (S321). A difference in a height direction (Z axis) with the background image stored in the background image storage unit 105 is obtained by the differentiator 106 to generate a person recognition image (S322). In the person recognition image, a point group of (X, Y) whose height exceeds a measurement error α is regarded as a contour of a person (S323). However, a contour not having a certain area is not regarded as a person. Further, a highest value in the contour is set to the height (Z) of the person, and a horizontal position thereof is set to a horizontal position (X, Y) of the person (S324). The height (Z) and the horizontal position (X, Y) of the person are used for generation of the life rhythm measurement data.

FIG. 3D is a flowchart illustrating a life rhythm measurement process (S330) in the TOF device 1. In this process, the life rhythm data conversion unit 12 performs conversion into a standing time, a sitting time, a lying time, an absence time, and a moving distance per minute based on a result of state determination (posture, presence/absence) or position information of the target person obtained in FIG. 3B to generate the life rhythm measurement data 15. Then, the data transmission unit 13 transmits the life rhythm measurement data 15 to the management device 2.

In the TOF sensor 10, 3D information (state or position information) of the target person is obtained every one second and converted into data of one minute, and thus integration is performed using a counter. For this reason, a state counter and a movement amount counter are initialized (S331), a 3D point group image is read every one second, and state determination for the target person described in FIG. 3B (S310) is performed (S332). Then, is added to a counter corresponding to a determined state (S333). For example, by using each of counters of the standing posture (Standing), the sitting posture (Sitting), the lying posture (Lying), absence (NoExist), when it is determined to be the sitting posture and the movement amount (Activity), “Sitting++” is set. Subsequently, a moving distance is calculated from previous coordinates (X0, Y0) and current coordinates (X1, Y1), and the movement amount counter is incremented (S334). That is, “Activity+”=SquareRoot ((X1−X0)**2+(Y1−Y0)**2)) is set.

S332 to S334 described above are repeated until 60 seconds has elapses (S335). After lapse of 60 seconds, life rhythm measurement data is created from the current state of the target person and a counter value (S336) and transmitted from the data transmission unit 13 to the management device 2 (S337). Returning to S331, life rhythm measurement data for a subsequent minute is created. Here, an example of transmission data (JSON format) is posted.

{“DeviceId”: “Tokkyo1”, “Time”: “2017-07-05T12:45:00+09:00”, “State”: “Standing”, “X”: 2610, “Y”: 1325, “Z”: 1237, “NoExistCount”: 5, “StandingCount”: 32, “SittingCount”: 15, “LyingCount”: 8, “Actibity”: 2969}.

In the present embodiment, information obtained by the TOF device 1 is limited to information on a 3D position of the measurement target person, and thus privacy is not violated. In addition, since a height and a size of an object can be determined based on the obtained information on the 3D position, a small animal is not erroneously recognized as a person. Meanwhile, the posture of the target person, that is, whether the target person is standing, sitting, lying down, or absent is detected from information obtained by the TOF device 1. Thus, it is possible to estimate a life rhythm such as an activity state, a rest state, or an absence state of the measurement target person by analyzing these pieces of information in chronological order.

Next, an operation on the management device 2 side will be described in detail. The data reception unit 21 receives the life rhythm measurement data 15 transmitted from the TOF device 1 every one minute, and the measurement data registration unit 22 deserializes received data in a JSON format for each item and registers deserialized minute-based data in the storage device 23 for each item.

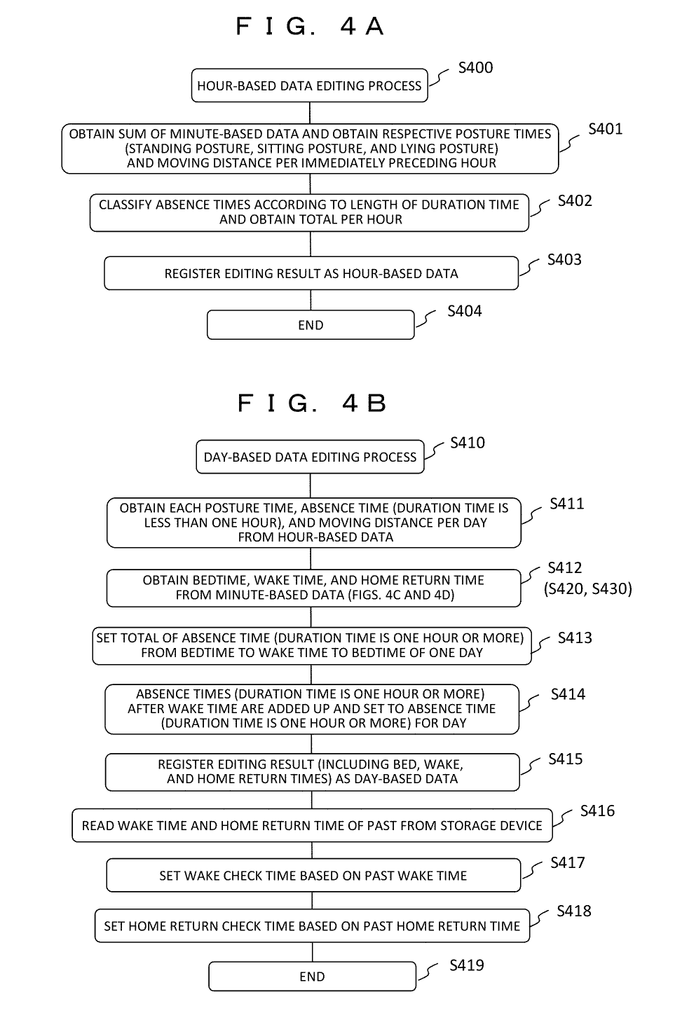

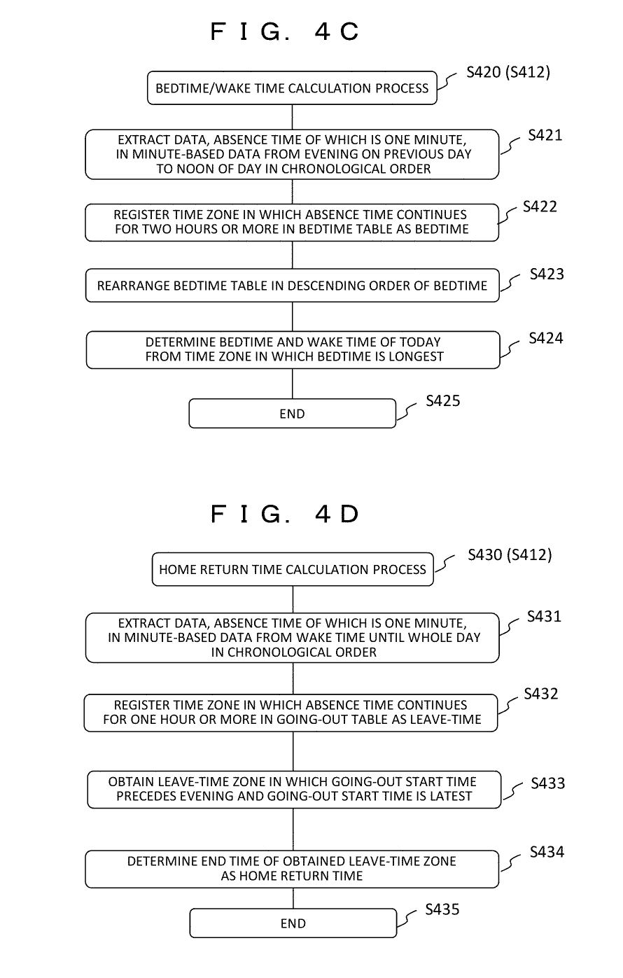

Next, various analysis operation flows of the life rhythm analysis unit 24 will be described with reference to FIG. 4A to FIG. 4D. The life rhythm analysis unit 24 aggregates and analyzes data accumulated in the storage device 23 to estimate the life rhythm of the measurement target person 3.

FIG. 4A is a flowchart illustrating an editing process (S400) from minute-based data to hour-based data. In this process, the minute-based data stored in the storage device 23 is edited into hour-based data for one hour and re-registered in the storage device.

A standing time, a sitting time, a lying time, and a moving distance of minute-based data from 0 minute to 59 minutes in an immediately preceding hour are read from the storage device 23, a sum thereof is obtained, and a standing time, a sitting time, a lying time, and a moving distance per hour are obtained (S401). In this instance, the standing, sitting, and lying times are calculated by simple addition. In addition, the moving distance is calculated by simple addition.

For example, absence times are classified into absence times of less than 10 minutes, 10 minutes to 20 minutes, 20 minutes to 30 minutes, 30 minutes to one hour, one hour to two hours, and two hours or more according to a length of a duration time of an absence time. Further, a total per hour is obtained for each duration time (S402). For this reason, a counter is used to classify lengths of absence times into respective duration times and aggregate the lengths. The editing result is re-registered in the storage device as hour-based data (S403).

FIG. 4B is a flowchart illustrating an editing process (S410) from hour-based data to day-based data. In this process, the hour-based data stored in the storage device 23 is edited into day-based data for one day and re-registered in the storage device. Further, a process of obtaining a bedtime, a wake time, and a home return time for one day from minute-based data of an absence time is performed.

A standing posture time, a sitting posture time, a lying posture time, an absence time (a duration time is less than one hour), and a moving distance of hour-based data from 0 o'clock to 23 o'clock on a previous day are read from the storage device, each sum thereof is obtained, and a standing posture time, a sitting posture time, a lying posture time, an absence time (a duration time is less than one hour), and a moving distance per day are obtained (S411). The standing posture, sitting posture, and lying posture times and the moving distance are calculated by simple addition.

Subsequently, a bedtime, a wake time, and a home return time of the target person are estimated from minute-based data (data of the absence time) (S412). This process will be separately described with reference to FIGS. 4C and 4D. A total of the absence time (the duration time is one hour or more) from the bedtime to the wake time is set to a bedtime of one day (S413). Absence times (duration time is one hour or more) after the wake time are added up and set to an absence time (duration time is one hour or more) of one day (S414). The editing result (including the bedtime, the wake time, and the home return time) are registered in the storage device 23 as day-based data (S415).

A wake time and a home return time of the past (for example, for two weeks) are read from the storage device 23 (S416). A multiple (26) of a standard deviation a of wake times for two weeks is added to an average value of the wake times for two weeks, and a timer is set as a wake check time (S417). Similarly, a multiple (26) of a standard deviation a of home return times for two weeks is added to an average value of the home return times for two weeks, and a timer is set as a home return check time (S418). Incidentally, when the wake check time precedes 8:00, the wake check time is set to 8:00. In addition, when the home return check time precedes 18:00, the home return check time is set to 18:00. When the wake check time and the home return check time set in the timer arrive, wake/home return check is performed by the wake/home return determination unit 26.

FIG. 4C is a flowchart illustrating a bedtime/wake time calculation process (S420), and specifically illustrates the process (S412) of FIG. 4B. First, in minute-based data from evening (18:00) on a previous day to noon (12:00) of a day, data, an absence time of which is one minute, is extracted in chronological order (S421). Further, a time zone in which the absence time continues for two hours or more is obtained and registered in a bedtime table as a bedtime (S422). In this instance, when the person is in the room within five minutes (example) per hour in the middle of the time zone (toilet in the middle of the night, etc.), this instance is ignored and the absence time is presumed to be continuous. When a plurality of bedtimes (absence times of two hours or more) is present, the bedtime table is rearranged in descending order of bedtime (S423). A bedtime and a wake time of today are determined from a time zone in which the bedtime is the longest (S424). Incidentally, when there is no absence time continuing for two hours or more in S422, it is presumed that the person is not in bed, and the bedtime and the wake time are not determined.

FIG. 4D is a flowchart illustrating a home return time calculation process (S430), and specifically illustrates the process (S412) of FIG. 4B. First, in minute-based data from the wake time (determined in S424) until a whole day (23:59), data, an absence time of which is one minute, is extracted in chronological order (S431). Further, a time zone in which the absence time continues for one hour or more is obtained and registered in a going-out table as a leave-time (S432). A leave-time zone in which a going-out start time precedes evening (18:00) and the going-out start time is the latest is obtained from the going-out table (S433). An end time of the obtained leave-time zone is determined as a home return time (S434). Incidentally, when there is no absence time continuing for one hour or more in S432, it is presumed that the person does not go out, and the home return time is not determined. In addition, when the person is not in the room after searching until 24:00, the home return time is not determined.

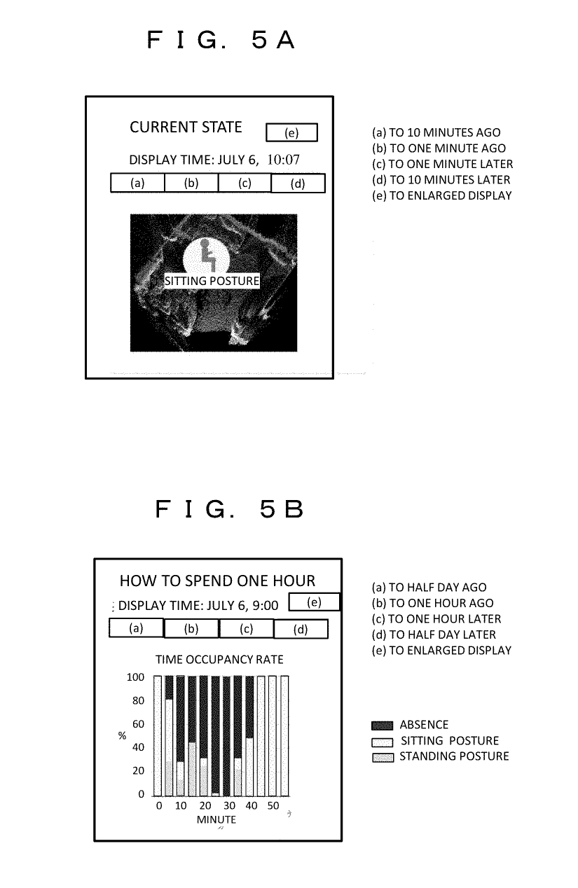

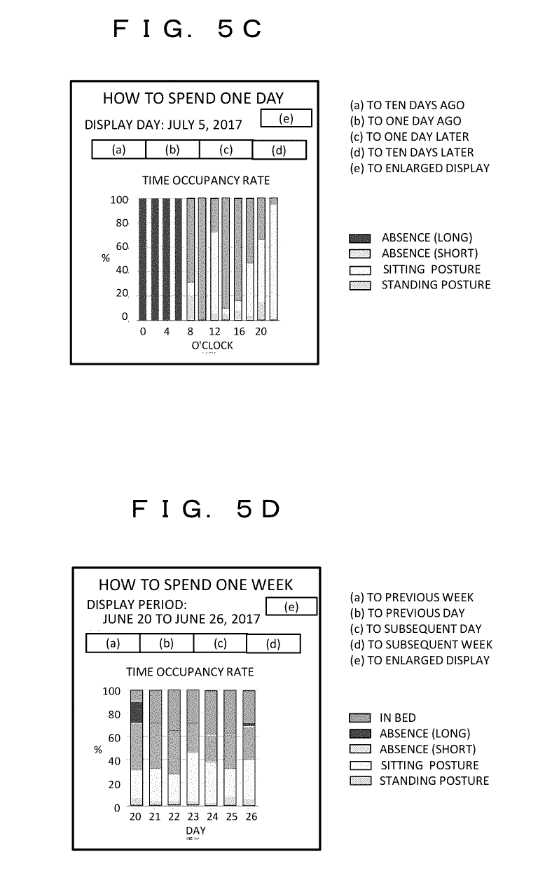

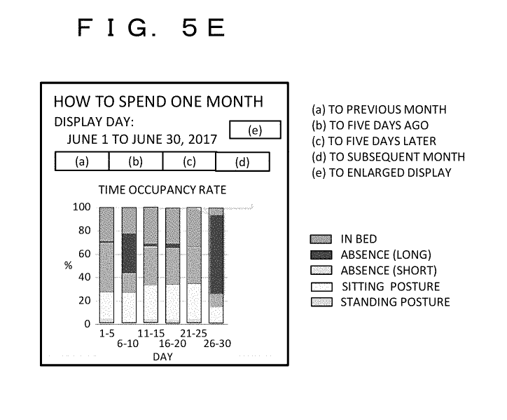

Next, a description will be given of provision of life rhythm information of the measurement target person 3 by the life rhythm providing unit 25. Upon receiving a call (login) from the measurement requester 4 via the Internet, the management device 2 provides the life rhythm information of the measurement target person 3 to the measurement requester 4 with reference to the storage device 23. Examples of a Web screen provided at this time are illustrated in FIG. 5A to FIG. 5E, and the measurement requester 4 may know the life rhythm of the measurement target person 3 by browsing the Web screen.

FIG. 5A is an example of a screen showing a current state of the measurement target person 3 in a room. The life rhythm providing unit 25 reads latest minute-based data from the storage device 23, and displays a position and a posture (“sitting” in this example) in the room having the target person therein on the Web screen. In the case of absence, “absence” is displayed. In addition, when a visitor other than the target person is present, the visitor is displayed together. In this way, it is possible to check a current state of the target person.

FIG. 5B is an example of a screen showing an hourly life rhythm of the measurement target person 3. Minute-based data within one hour designated by the measurement requester 4 is read, and accumulation times of the standing posture/sitting posture/lying posture/absence are displayed in a 100% stacked bar graph on the Web screen in increments of five minutes. In addition, the moving distance of the target person is displayed in a bar graph. In this way, it is possible to check activity of the target person every hour.

FIG. 5C is an example of a screen showing a daily life rhythm of the measurement target person 3. Hour-based data within one designated day is read, and accumulation times of the standing posture/sitting posture/lying posture/absence are displayed in a 100% stacked bar graph on the Web screen in increments of two hours. Absence times are displayed by being classified into less than 10 minutes, less than 20 minutes, less than 30 minutes, less than one hour, less than two hours, and two hours or more depending on the continuous length. In addition, the moving distance of the target person is displayed in a bar graph. In this way, it is possible to check activity of the target person every day. In other words, it is possible to infer times of wake, going out, returning home, and going to bed and the amount of activity (movement amount) on the corresponding day of the target person.

FIG. 5D is an example of a screen showing a weekly life rhythm of the measurement target person 3. Day-based data of a designated week is read, and accumulation times of the standing posture/sitting posture/lying posture/absence are displayed in a 100% stacked bar graph on the Web screen in increments of one day. Absence times are displayed by being classified into less than 10 minutes, less than 20 minutes, less than 30 minutes, less than one hour, less than two hours, two hours or more, and a bedtime depending on the continuous length. In addition, the moving distance of the target person is displayed in a bar graph. In this way, it is possible to check an active day and a rest day with regard to activity of the target person every week.

FIG. 5E is an example of a screen showing a monthly life rhythm of the measurement target person 3. Day-based data of a designated month is read, an average value is taken in units of five days, and accumulation times of the standing posture/sitting posture/lying posture/absence are displayed in a 100% stacked bar graph on the Web screen. Absence times are displayed by being classified into less than 10 minutes, less than 20 minutes, less than 30 minutes, less than one hour, less than two hours, two hours or more, and a bedtime depending on the continuous length. In addition, the moving distance of the target person is displayed in a bar graph. In this way, it is possible to check activity of the target person every month. Further, it is possible to read a change in sleeping time or activity amount by comparing an average value of one arbitrary past month with current data. Besides, it is possible to provide a similar display screen for a desired period such as a yearly life rhythm.

Subsequently, when it is determined that a latest life rhythm of the target person 3 is different from a past life rhythm, or when the falling determination unit 27 determines that the target person has fallen, the wake/home return determination unit 26 issues a notification of an alarm from the mail transmission unit 28 to the measurement requester 4.

First, a description will be given of a wake check/home return check process for the measurement target person 3 by the wake/home return determination unit 26. A wake check time and a home return check time are set in the timer by the process (S417 and S418) of FIG. 4B. When the set time arrives, the wake/home return determination unit 26 performs the subsequent process.

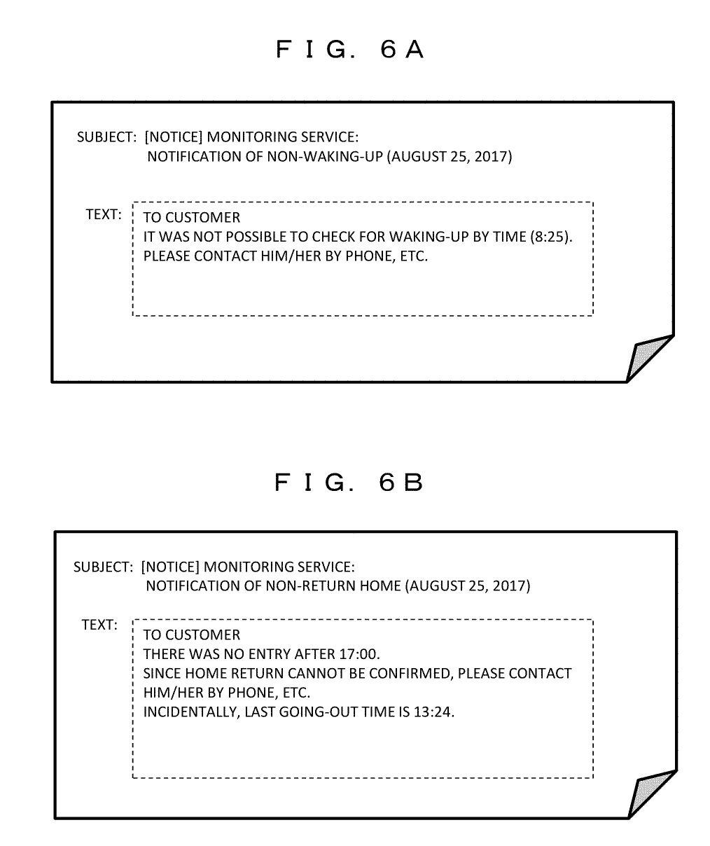

In wake check, minute-based data of the latest five minutes is read. Further, when the target person is in the room for one second or more, it is determined that the person has woken up. Alternatively, minute-based data of the latest one hour is read. Further, when the target person is in the room for five minutes or more, it is determined that the person has woken up. When presence in the room in either case may not be confirmed, a “non-waking-up mail” is transmitted to the measurement requester 4 via the mail transmission unit 28. FIG. 6A is a diagram illustrating a content example of the non-waking-up mail.

In home return check, minute-based data of the latest one hour is read. Further, when the target person is in the room for one second or more, it is determined that the person has returned home. When presence in the room may not be confirmed, a “non-home return mail” is transmitted to the measurement requester 4 via the mail transmission unit 28. FIG. 6B is a diagram illustrating a content example of the non-home return mail.

Further, the falling determination unit 27 performs a fall check process for the measurement target person 3. In fall check, a posture and a position of the target person are monitored based on the life rhythm measurement data 15 transmitted from the TOF device 1 every one minute, and an abnormality is detected when the target person lies down at a position at which the target person normally does not lie down. That is, when the received life rhythm measurement data indicates posture=lying posture, and a position (X, Y) thereof corresponds to a position at which the target person has not lain down in the past, it is determined that there is a risk that the target person has fallen, and a falling time counter is activated. Then, a time at which measurement data indicating the same position in the same posture continues is measured by the falling time counter, and it is determined to be “falling” at a point in time when a duration time has passed, for example, five minutes. Then, a mail sentence reporting falling is transmitted to the measurement requester 4 via the mail transmission unit 28. Even though mail content in this case is omitted, the same format as that of FIGS. 6A and 6B is adopted.

In the present embodiment, a scheme of notifying the measurement requester 4 when an abnormality is detected is performed by mail transmission. However, another communication means such as a telephone line may be used.

A description has been given of a configuration and an operation of the life rhythm measurement system of the present embodiment, and there are merits below when compared to a conventional monitoring system. (1) A reason for absence can be inferred from absence information. When the TOF sensor is installed in the living room, etc., it is possible to measure an absence time in the living room. Since lengths of absence times are classified into a plurality of stages (less than 10 minutes, less than 20 minutes, less than 30 minutes, less than one hour, less than two hours, and two hours or more), the measurement requester 4 or a person involved may surmise that the target person went to the toilet or the kitchen, went to a neighborhood, or went out. In addition, since the absence time is measured throughout 24 hours, it is possible to estimate the bedtime and the wake time.

(2) It is possible to measure the activity amount of the measurement target person. It is possible to measure the movement amount in the living room by tracking 3D distance information (position information) of the target person. It is possible to estimate an active time zone and a resting time zone by obtaining the movement amount per unit time.

(3) An abnormality can be rapidly detected. The concern for the target person is whether the person wakes up at the usual time in the morning, whether the person returns home in the evening, and whether the person falls due to illness or injury. In a conventional door sensor and electric kettle sensor, when a state largely deviating from the usual usage method continues, it is determined as an abnormality. For this reason, it takes several hours from an occurrence of an abnormality until the abnormality is detected. On the other hand, in the present embodiment, since it is possible to know a current living condition of the target person from data of the TOF sensor, it is determined as an abnormality at the time of being delayed from a normal behavior pattern of the past (a wake time, a home return time) by a predetermined amount. Therefore, an abnormality can be detected at the same level as a time required for a living person to sense the abnormality.

In addition, as for the fall check, detection can be performed from the fact that the target person is lying at a different position from usual.

(4) The measurement target person may not wear a measuring instrument. For example, there is a wearable activity meter as an instrument for measuring accurate physical information of the target person. However, this instrument is unsuitable for the elderly person since the target person needs to wear the instrument at all times. On the other hand, TOF sensor may not be worn, and thus may be accepted even for the elderly person without resistance.

The invention is not limited to the embodiment described above, and includes various modifications. The above-described embodiment has been described in detail in order to describe the invention in an easy-to-understand manner, and may not have all the configurations described. In addition, numerical values such as time widths mentioned in the embodiment are merely examples, and may be appropriately changed and set according to a use environment.

What is claimed is:

1. A life rhythm measurement system for measuring a life rhythm of a measurement target person, the life rhythm measurement system comprising:
a distance measuring device that measures three-dimensional (3D) information of the measurement target person present in a specific living space; and
a management device that aggregates the 3D information measured by the distance measuring device for each unit time, estimates the life rhythm of the measurement target person from a 3D data for each time, and accumulates the estimated life rhythm in a storage device,
wherein the management device provides information on the life rhythm of the measurement target person accumulated in the storage device to a measurement requester.

2. The life rhythm measurement system according to claim 1,
wherein the distance measuring device determines whether a posture of the measurement target person corresponds to a standing posture, a sitting posture, or a lying posture as the 3D information, and transmits determined posture information to the management device, and
the management device calculates a ratio of a time during which the posture of the measurement target person corresponds to the standing posture, the sitting posture, or the lying posture per unit period as the life rhythm, and accumulates the calculated ratio in the storage device.

3. The life rhythm measurement system according to claim 2,
wherein the distance measuring device further calculates a movement amount of the measurement target person as the 3D information, and transmits the calculated movement amount to the management device, and
the management device further calculates a movement amount per unit period from the movement amount of the measurement target person as the life rhythm, and accumulates the calculated movement amount in the storage device.

4. The life rhythm measurement system according to claim 2,
wherein the distance measuring device further determines presence/absence of the measurement target person as the 3D information, and transmits determined absence information to the management device, and
the management device further calculates an absence time per unit period from the absence information of the measurement target person as the life rhythm, and accumulates the calculated absence time in the storage device.

5. The life rhythm measurement system according to claim 4, wherein the management device estimates a bedtime, a wake time, or a home return time of the measurement target person from the calculated absence time as the life rhythm, and accumulates the estimated time in the storage device.

6. The life rhythm measurement system according to claim 1, wherein the management device compares a current life rhythm of the measurement target person with an average value of past life rhythms accumulated in the storage device, and notifies the measurement requester of an abnormality when the current life rhythm is different from the average value by a predetermined amount or more.

7. The life rhythm measurement system according to claim 1, wherein the distance measuring device measures a 3D distance to the measurement target person by a flight time of light, and calculates a position and a shape of the measurement target person.

8. A life rhythm measurement method of measuring a life rhythm of a measurement target person, the life rhythm measurement method comprising:
a step of measuring 3D information of the measurement target person present in a specific living space;
a step of aggregating the measured 3D information for each unit time, estimating the life rhythm of the measurement target person from a 3D data for each time, and accumulating the estimated life rhythm in chronological order; and
a step of providing information on the accumulated life rhythm of the measurement target person to a measurement requester.

9. The life rhythm measurement method according to claim 8, further comprising
a step of comparing a current life rhythm of the measurement target person with an average value of accumulated past life rhythms, and notifying the measurement requester of an abnormality when the current life rhythm is different from the average value by a predetermined amount or more.
